(19) United States
(12) Patent Application Publication
(10) Pub. No.: US 2007/0078474 A1
(43) Pub. Date: Apr. 5, 2007

(54) Single-use lancet device

(76) Inventor: Yong Pil Kim, Seoul (KR)

Correspondence Address: MERCHANT & GOULD PC, P.O. BOX 2903, MINNEAPOLIS, MN, 55402-0903, US

(21) Appl. No.: 11/243,770

(22) Filed: Oct. 5, 2005

(51) Int. Cl. A61B 17/32

(52) U.S. Cl. 606/181

(57) ABSTRACT

A single-use lancet device comprises a main body having a shooting hole and a fixing end, a first elastic member inserted in the main body, a wing plate catching member disposed in the main body while being caught by the fixing end and having a plurality of catching protrusions formed at the front end thereof, an actuating member disposed at the rear of the wing plate catching member such that a lancet is fixed to the front end of the actuating member, the actuating member having a wing plate and a stopping protrusion, the diameters of which are gradually decreased from the rear ends to the front ends of the wing plate and the stopping protrusion, the wing plate being caught by the catching protrusions, a second elastic member disposed at the rear of the actuating member, and a push member disposed at the rear part of the main body for applying pressure to the actuating member through the second elastic member when a user pushes the push member forward.

### BACKGROUND OF THE INVENTION

1. Field of the Invention

The present invention relates to a lancet device used to collect blood from a human body for the purpose of medical treatment, and, more particularly, to a single-use lancet device wherein reuse of the single-use lancet is prevented, the number of components constituting the single-use lancet device is decreased, and the assembly efficiency of the single-use lancet device is improved.

2. Description of the Related Art

Generally, blood is collected, as occasion demands, to execute health examinations or treat patients. When a large amount of blood is needed, a syringe is used to collect the blood. When a small amount of blood is needed, for example, when the sugar content contained in blood of a diabetic is examined or a blood type test is performed, on the other hand, a lancet device is used to collect the blood.

FIG. 1A is a perspective view illustrating a conventional single-use lancet device, and FIG. 1B is a sectional view of the conventional single-use lancet device. As shown in FIGS. 1A and 1B, a lancet 32 is fixed to the front end of an actuating member 30. The rear end of the actuating member 30 is coupled with an elastic member 40. The actuating member 30 is provided at the rear part thereof with a protrusion 32, which is caught by a lower inclined end 22 integrally formed with a push member 20, which is disposed at the outside of a main body 10 and is inserted in the main body 10. When the push member 20 is pushed, the lower inclined end 22 is tilted, by which the locked state of the actuating member 30 is released. As a result, the actuating member 30 extends forward by the elastic force accumulated in the elastic member 40, and therefore, the lancet 32 fixed to the front end of the actuating member 30 is shot outward from the main body 10. In this conventional single-use lancet device, however, when a user retracts the actuating member 30, the protrusion 32 of the actuating member 30 is moved backward while pushing the lower inclined end 22 of the push member 20. After the actuating member 30 is retracted to its initial position, the lower inclined end 22 of the push member 20 is returned to its original state, and therefore, the actuating member is automatically locked by the lower inclined end 22 of the push member 20 while the elastic force is accumulated in the elastic member 40. Consequently, reuse of the single-use lancet device is possible. When the used lancet device is reused, however, sanitary problems, such as contagion, may occur, since the used lancet is stained with blood.

In order to solve the above-mentioned problems, the applicant of the present application has developed a single-use lancet device, which is disclosed in Korean Registered Utility Model Nos. 20-0296733 (published on Dec. 5, 2002) and 20-0315777 (published on Jun. 11, 2003). Reuse of the single-use lancet device disclosed in the above-mentioned registered utility models is prevented. However, the structure of the single-use lancet device is complicated, and the number of components constituting the single-use lancet device is great. Consequently, the manufacturing costs of the single-use lancet device are increased.

### SUMMARY OF THE INVENTION

Therefore, the present invention has been made in view of the above problems, and it is an object of the present invention to provide a single-use lancet device wherein a lancet is not retracted once the lancet is shot such that reuse of the single-use lancet is prevented, the number of components constituting the single-use lancet device is decreased, and the assembly efficiency of the single-use lancet device is improved.

In accordance with the present invention, the above and other objects can be accomplished by the provision of a single-use lancet device including a lancet, which is shot by an elastic force accumulated in an elastic member to penetrate into a skin of a user such that bleeding is accomplished from the skin, wherein the lancet device comprises: a main body formed in the shape of a hollow cylinder, the main body having a shooting hole formed at the front end thereof and a fixing end formed at the inner circumferential surface thereof adjacent to a front part thereof; a first elastic member inserted in the front part of the main body; a wing plate catching member formed in the shape of a hollow cylinder, the wing plate catching member being disposed in the main body while being caught by the fixing end, the wing plate catching member having a plurality of catching protrusions formed at the front end thereof while being protruded inward along the inner circumferential surface thereof; an actuating member disposed at the rear of the wing plate catching member, the lancet being fixed to the front end of the actuating member, the actuating member having a truncated-conical wing plate and a truncated-conical stopping protrusion formed at the outer circumferential surface thereof while the wing plate and the stopping protrusion are spaced a predetermined distance from each other, the wing plate and the stopping protrusion being constructed such that the diameters of the wing plate and the stopping protrusion are gradually decreased from the rear ends to the front ends of the wing plate and the stopping protrusion, the wing plate being caught by the catching protrusions; a second elastic member disposed at the rear of the actuating member; and a push member disposed at the rear part of the main body, while being protruded outward from the main body, for applying pressure to the actuating member through the second elastic member when a user pushes the push member forward, whereby, when a predetermined force is applied to the actuating member from the push member, the wing plate can pass forward through the wing plate catching member while the catching protrusions are bent, but the wing plate cannot pass backward through the wing plate catching member. Consequently, the lancet is not retracted into the main body once the lancet is shot, and therefore, reuse of the single-use lancet is prevented. Furthermore, the number of components constituting the single-use lancet device is decreased, and therefore, the assembly efficiency of the single-use lancet device is improved.

Preferably, the main body is provided at the inner circumferential surface of the front part thereof with protrusions, which are protruded to a predetermined height while being parallel with each other, and the wing plate and the stopping protrusion are provided at predetermined positions thereof with flat surface parts, which are diametrically opposite to each other, the flat surface parts of the wing plate and the stopping protrusion corresponding to the protrusions of the main body. Consequently, rotation of the actuating member in the main body is effectively prevented.

Preferably, the single-use lancet device further comprises: a protective cap inserted in the shooting hole of the main body, the protective cap being provided at one end thereof with a groove having a predetermined depth, into which the lancet can be inserted. Consequently, the lancet is effectively prevented from being infected and brought into direct contact with the skin of the user.

Preferably, the main body is provided at the outside thereof with a disc-shaped grip part, which is protruded along the outer circumferential surface of the main body. Consequently, manipulation of the single-use lancet device is easy and convenient.

Preferably, the grip part is provided with a plurality of a flat surface part, which is protruded along the outer circumferential surface of the grip part. Consequently, the single-use lancet device cannot roll over.

### BRIEF DESCRIPTION OF THE DRAWINGS

The above and other objects, features and other advantages of the present invention will be more clearly understood from the following detailed description taken in conjunction with the accompanying drawings, in which:

FIG. 1A is a perspective view illustrating a conventional single-use lancet device;

FIG. 1B is a sectional view of the conventional single-use lancet device;

FIG. 2 is a perspective view illustrating a single-use lancet device according to the present invention;

FIG. 3 is an exploded perspective view of the single-use lancet device according to the present invention;

FIG. 4 is a sectional view taken along line A-A′ of FIG. 3; and

FIG. 5A is a sectional view of the single-use lancet device according to the present invention before the lancet is shot;

FIG. 5B is a sectional view of the single-use lancet device according to the present invention when the lancet is shot; and

FIG. 5C is a sectional view of the single-use lancet device according to the present invention after the lancet is shot.

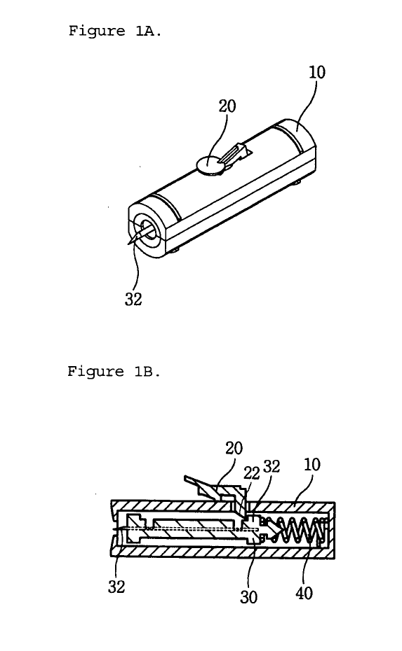

### DESCRIPTION OF THE PREFERRED EMBODIMENTS

Now, a preferred embodiment of the present invention will be described in detail with reference to the accompanying drawings.

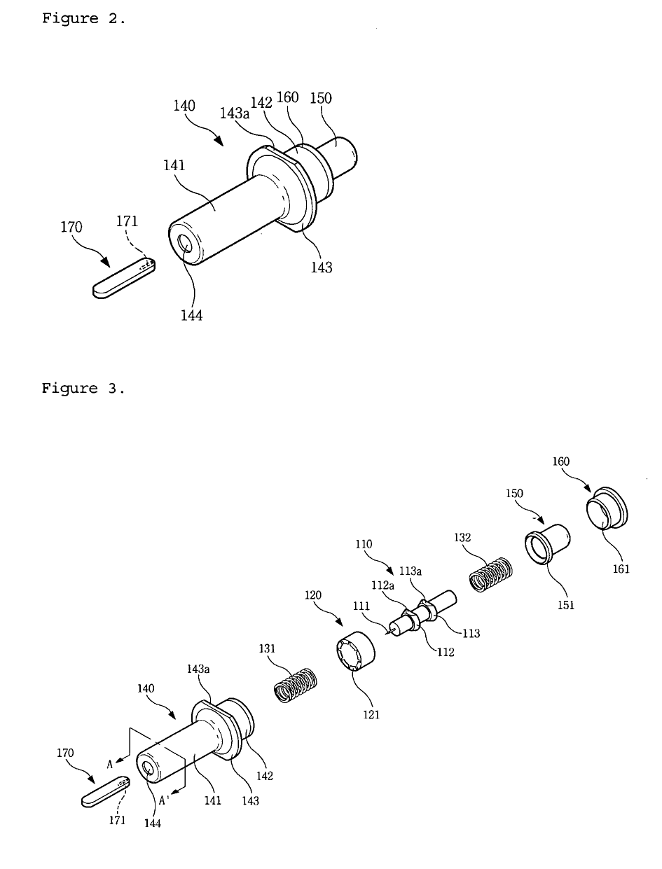

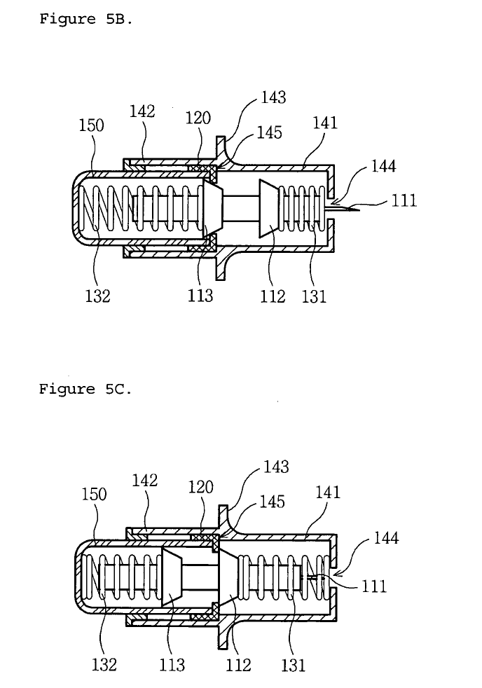

FIG. 2 is a perspective view illustrating a single-use lancet device according to the present invention. As shown in FIG. 2, an upper case 160 is coupled with the rear end of a main body 140, and a push member 150 is inserted through a circular hole 161 (see FIG. 3) formed at the upper case 160. The main body 140 is formed in the shape of a hollow cylinder, in which components of the lancet device are disposed. The main body 140 comprises a front main body 141, a rear main body 142, and a grip part 143. The diameter of the rear main body 142 is greater than that of the front main body 141. The main body 140 is manufactured such that the main body 140 has a predetermined thickness. For this reason, a fixing end 145 (see FIG. 5A) having a relatively small front-side inner diameter is formed at the boundary between the front main body 141 and the rear main body 142 inside the main body 140.

The grip part 143 is formed in the shape of a disc at the outer circumferential surface of the main body 140 at the boundary between the front main body 141 and the rear main body 142. The grip part 143 is sized such that the index and middle fingers of a user can be placed on the front end of the grip part 143 when the user holds the lancet device to use the lancet device. And the grip part 143 is provided with a plurality of a flat surface part 143a, which is protruded along the outer circumferential surface of the grip part 143. Consequently, the single-use lancet device cannot roll over on a floor.

The upper case 160 is coupled with the rear end of the main body 140. The circular hole 161 (see FIG. 3), which has a predetermined diameter, is formed through the center of the upper case 160 such that the push member 150 is inserted through the circular hole 161 of the upper case 160. The push member 150 is protruded outward from the main body 140 through the circular hole 161 of the upper case 140 such that the user can easily and conveniently push the push member 150 by the thumb when the lancet device is used.

FIG. 3 is an exploded perspective view of the single-use lancet device according to the present invention. At the front end of the main body 140 is formed a shooting hole 144, through which a lancet 111 extends outward from the main body 140 and then penetrates into the skin of the user when the lancet 111 is shot.

When the single-use lancet device according to the present invention is not used, the lancet 111 may be protected by a protective cap 170. The protective cap 170 is inserted in the shooting hole 144 of the main body 140. The protective cap 170 is provided at one end thereof with a groove 171 having a predetermined depth, into which the lancet 111 can be inserted. The protective cap 170 serves to prevent the lancet 111 from being infected and to prevent the lancet 111 from being brought into direct contact with the skin of the user when the lancet 111 is accidentally shot and the lancet 111 extends outward from the main body 140. As shown in FIGS. 2 and 3, the protective cap 170 is formed in a flat shape, by which the user can easily hold and remove the protective cap 170.

In the main body 140 are mounted a first elastic member 131, a wing plate catching member 120, an actuating member 110, a second elastic member 132, and the push member 150 in order from the front end to the rear end of the main body 140.

The first elastic member 131 is disposed in the front main body 141 after the first elastic member 131 is inserted into the main body 140. The actuating member 110 is inserted in the first elastic member 131 while the front end of a wing plate 112 formed at the actuating member 110 is not inserted in the first elastic member 131. Consequently, the first elastic member 131 is compressed when the actuating member 110 is moved forward, and is then extended by its elastic force to move the actuating member 110 backward.

The wing plate catching member 120 is formed in the shape of a hollow cylinder. At the front end of the wing plate catching member 120 are formed a plurality of catching protrusions 121, which are protruded inward along the inner circumferential surface of the wing plate catching member 120. The catching protrusions 121 are made of an elastic material, and therefore, the catching protrusions 121 are bent when a predetermined external force is applied to the catching protrusions 121. The outer diameter of the wing plate catching member 120 is slightly less than the inner diameter of the rear main body 142 while the outer diameter of the wing plate catching member 120 is slightly greater than the inner diameter of the front main body 141. As a result, the wing plate catching member 120 is disposed in the main body 140 while being caught by the fixing end 145. The actuating member 110 is disposed at the rear of the wing plate catching member 120, and the wing plate 112 of the actuating member 110 is caught by the catching protrusions 121 of the wing plate catching member 120.

The actuating member 110 is formed in the shape of an elongated cylinder. The lancet 111 is fixed to the front end of the actuating member 110. The wing plate 112 is formed at the outer circumferential surface of the actuating member 110. The wing plate 112 is constructed such that the diameter of the wing plate 112 is gradually decreased from the rear end to the front end of the wing plate 112. At the outer circumferential surface of the actuating member 110 is also formed a stopping protrusion 113, which is spaced a predetermined distance from the wing plate 112. The stopping protrusion 113 is formed in the same shape as the wing plate 112, and the size of the stopping protrusion 113 is equal to or greater than that of the wing plate 112.

As described above, the diameter of the wing plate 112 at the rear end thereof is greater than that of the wing plate 112 at the front end thereof, and the catching protrusions 121 of the wing plate catching member 120 are made of the elastic material such that the catching protrusions 121 can be bent. Consequently, when the actuating member 110 is moved forward, a force greater than the elasticity of the catching protrusions 121 is applied to the wing plate 112, and therefore, the wing plate passes forward through the catching protrusions 121. After the wing plate is moved forward through the catching protrusions 121, however, the wing plate 112 cannot pass backward through the catching protrusions 121. At predetermined positions of the wing plate 112 are formed flat surface parts 112a, which are diametrically opposite to each other. Specifically, the flat surface parts 112 are formed by cutting the edges of the wing plate 112 to a predetermined depth in the longitudinal direction of the actuating member 110. Similarly, flat surface parts 113a are formed at predetermined positions of the stopping protraction 113 while the flat surface parts 113a are diametrically opposite to each other. The flat surface parts 113a are also formed by cutting the edges of the stopping protraction 113 to a predetermined depth in the longitudinal direction of the actuating member 110.

The second elastic member 132 is disposed inside the main body 140 while the actuating member 110 is inserted in the second elastic member 132. The front end of the second elastic member 132 is brought into contact with the rear end of the stopping protrusion 113, and the rear end of the second elastic member 132 is brought into contact with the push member 150.

The push member 150 is formed in the shape of a hollow cylinder, the rear end of which is closed. The second elastic member 132 is inserted in the push member 150. The push member 150 is also inserted in the main body 140. After the upper case 160 is coupled with the main body 140, the push member 150 is protruded out of the main body 140 through the circular hole 161 of the upper case 160. At the front end of the push member 150 is formed a flange 151, which is caught by the front end of the upper case 150 such that the push member 150 is separated from the main body 140. The rear end of the push member 150 is pushed by the thumb of the user when the lancet device according to the present invention is used. For this reason, the rear end of the push member 150 is preferably flat such that the pushing operation of the push member 150 is easily and conveniently accomplished.

The upper case 160 is formed in the shape of a hollow cylinder. The upper case 160 is coupled with the rear end of the main body 140 such that the push member 150 and other components of the lancet device are securely disposed in the main body 140. As described above, the circular hole 161 is formed at the upper case 160, and therefore, the push member 150 extends outward from the main body 140.

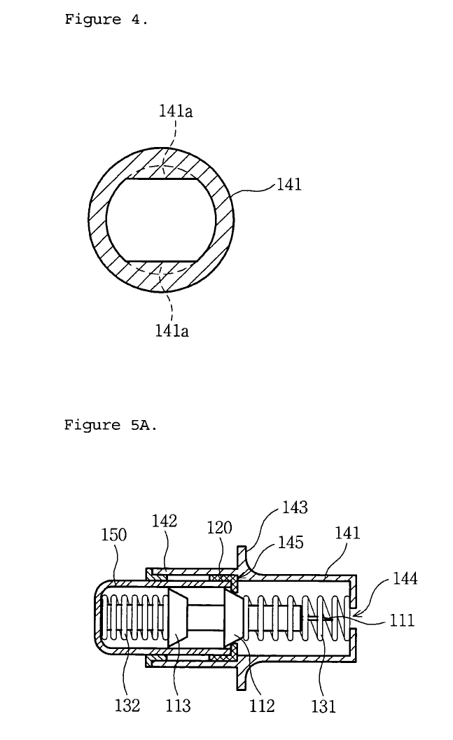

FIG. 4 is a sectional view taken along line A-A′ of FIG. 3, illustrating the inner shape of the front main body 141. At predetermined positions of the inner circumferential surface of the front main body 141 are formed protrusions 141a, which are protruded to a predetermined height while being parallel with each other. The protrusions 141a correspond to the flat surface parts 112a of the wing plate 112 and the flat surface parts 113a of the stopping protrusion 113. In this way, the protrusions 141a are formed at the inner circumferential surface of the front main body, and the flat surface parts 112a and 113a are formed at the wing plate 112 and the stopping protrusion 113. Consequently, when the actuating member 110 is inserted in the front main body 141, the actuating member 110 cannot be rotated.

FIGS. 5A to 5C illustrate the state of the single-use lancet device according to the present invention before the lancet is shot, when the lancet is shot, and after the lancet is shot. The operational principle of the single-use lancet device according to the present invention will be described hereinafter in detail with reference to the drawings.

Referring to FIGS. 3 and 5A, when the user pushes the push member 150, the second elastic member 132 disposed in the push member 150 is compressed. As the user continues to apply an external force to the push member 150, the second elastic member 132 is further compressed. At this time, the actuating member is moved forward by the elastic force of the second elastic member 132. As a result, the wing plate 112 of the actuating member 110 is caught by the wing plate catching member 120, which is also caught by the fixing end 145 inside the main body 140. FIG. 5A illustrates that the external force is applied to the push member 150 until the rear end of the second elastic member 132 is close by the rear end of the actuating member 110.

Referring to FIG. 5B, when the user further applies the external force to the push member 150 at the state shown in FIG. 5A, the external force applied by the user is transmitted to the actuating member 110 by the elastic force of the second elastic member 132. If the elastic modulus of the second elastic member 132 is small, the push member 150 is brought into direct contact with the rear end of the actuating member 110, and therefore, the external force applied by the user is directly transmitted to the actuating member 110. Since the catching protrusions 121 of the wing plate catching member 120 are made of the elastic material as described above, the catching protrusions 121 is bent when a predetermined external force is applied to the catching protrusions 121. Consequently, the wing plate 112, which is caught by the catching protrusions 121, passes through the bent catching protrusions 121. At this time, the actuating member is instantaneously shot by the elastic force accumulated in the second elastic member 132 or the external force of the user. As a result, the lancet 111 fixed to the front end of the actuating member 110 is also shot forward together with the actuating member 110. Consequently, the lancet 111 extends outward from the main body 140 through the shooting hole 144, and then penetrates into the skin of the user.

When the lancet 111 is shot outward from the main body 140, the extending length of the lancet 111 extending outward from the main body 140, i.e., the penetrating depth of the lancet 111 penetrating into the skin of the user, depends on the distance between the wing plate 112 and the stopping protrusion 113. The wing plate catching member 120 is caught at the boundary between the rear main body 142 and the front main body 141, i.e., at the inside of the main body 140 where the grip part 143 is formed, and the shape of the stopping protrusion 113 is nearly identical to that of the wing plate 112. Consequently, the stooping protrusion 113 is brought into contact with the wing plate catching member 120 in the course of the forward-shooting operation of the actuating member 110. As soon as the stooping protrusion 113 is brought into contact with the wing plate catching member 120, the movement of the stopping member 110 is stopped, and therefore, the lancet 111 does not extend outward from the main body 140 any more. As a result, the lancet 111 can be shot a distance between the wing plate 112 and the stopping protrusion 113 from the position shown in FIG. 5A to the position shown in FIG. 5B. As shown in FIG. 5B, the first elastic member 131 is compressed by the actuating member 110 moved to the front of the main body 140, and therefore, the elastic force is accumulated in the first elastic member 131.

Referring to FIG. 5C, immediately after the lancet 111 is shot outward from the main body 140, the first elastic member 131, which is compressed, and therefore, accumulates the elastic force, is extended by the elastic force to push the actuating member 110 backward. By this action of the first elastic member 131, the lancet 111 does not remain protruded outward from the main body 140 but is immediately retracted into the main body 140 after the lancet device is used. Consequently, safety of the lancet device is accomplished when in use. Furthermore, since the diameter of the wing plate 112 is gradually decreased from the rear end to the front end of the wing plate 112, the wing plate 112 of the actuating member 110 moved backward by the elastic force of the first elastic member 131 is caught by the wing plate catching member 120. As a result, the further backward-movement of the actuating member 110 is prevented.

The single-use lancet device according to the present invention is embodied by designing the shape of the wing plate 112 as described above. Conventional lancet devices have a problem in that the user may freely insert the lancet into the main body, and therefore, the lancet devices may be reused. However, this problem of the conventional lancet devices is easily solved by the structural characteristics of the wing plate 112 and the wing plate catching member 120 of the lancet device according to the present invention. Since the wing plate 112 is constructed such that the diameter of the wing plate 112 is gradually decreased from the rear end to the front end of the wing plate 112, i.e., the wing plate 112 is formed in the shape of a truncated cone, the wing plate 112 can easily pass forward through the catching protrusions 121 of the wing plate catching member 120. However, the wing plate 112 cannot pass backward through the catching protrusions 121 of the wing plate catching member 120 after the forward-movement of the wing plate 112. This operation of the wing plate 112 is performed based on the principle of an inclined plane. In this way, the present invention is embodied through simple change of the structure of the lancet device.

Furthermore, the structure of the lancet device according to the present invention is simple as shown in FIG. 3, and the lancet device according to the present invention is easily assembled by inserting the first elastic member 131, the wing plate catching member 120, the actuating member 110, the second elastic member 132, the push member 150 and the upper case 160 into the main body in consecutive order.

As apparent from the above description, the single-use lancet device according to the present invention has the structure in which the lancet cannot be retracted into the main body once the lancet is shot outward. Consequently, the present invention has the effect of preventing reuse of the lancet device. Furthermore, the number of components of the single-use lancet device according to the present invention is decreased. Consequently, the present invention has the effect of improving the assembly efficiency of the single-use lancet device.

Although the preferred embodiment of the present invention have been disclosed for illustrative purposes, those skilled in the art will appreciate that various modifications, additions and substitutions are possible, without departing from the scope and spirit of the invention as disclosed in the accompanying claims.

1. A single-use lancet device including a lancet, which is shot by an elastic force accumulated in an elastic member to penetrate into a skin of a user such that bleeding is accomplished from the skin, wherein the lancet device comprises:
a main body formed in the shape of a hollow cylinder, the main body having a shooting hole formed at the front end thereof and a fixing end formed at the inner circumferential surface thereof adjacent to a front part thereof;
a first elastic member inserted in the front part of the main body;
a wing plate catching member formed in the shape of a hollow cylinder, the wing plate catching member being disposed in the main body while being caught by the fixing end, the wing plate catching member having a plurality of catching protrusions formed at the front end thereof while being protruded inward along the inner circumferential surface thereof;
an actuating member disposed at the rear of the wing plate catching member, the lancet being fixed to the front end of the actuating member, the actuating member having a truncated-conical wing plate and a truncated-conical stopping protrusion formed at the outer circumferential surface thereof while the wing plate and the stopping protrusion are spaced a predetermined distance from each other, the wing plate and the stopping protrusion being constructed such that the diameters of the wing plate and the stopping protrusion are gradually decreased from the rear ends to the front ends of the wing plate and the stopping protrusion, the wing plate being caught by the catching protrusions;
a second elastic member disposed at the rear of the actuating member; and
a push member disposed at the rear part of the main body, while being protruded outward from the main body, for applying pressure to the actuating member through the second elastic member when a user push the push member forward,
whereby, when a predetermined force is applied to the actuating member from the push member, the wing plate can pass forward through the wing plate catching member while the catching protrusions are bent, but the wing plate cannot pass backward through the wing plate catching member.

2. The lancet device as set forth in claim 1, wherein
the main body is provided at the inner circumferential surface of the front part thereof with protrusions, which are protruded to a predetermined height while being parallel with each other, and
the wing plate and the stopping protrusion are provided at predetermined positions thereof with flat surface parts, which are diametrically opposite to each other, the flat surface parts of the wing plate and the stopping protrusion corresponding to the protrusions of the main body.

3. The lancet device as set forth in claim 1, further comprising:
a protective cap inserted in the shooting hole of the main body, the protective cap being provided at one end thereof with a groove having a predetermined depth, into which the lancet can be inserted.

4. The lancet device as set forth in claim 1, wherein the main body is provided at the outside thereof with a disc-shaped grip part, which is protruded along the outer circumferential surface of the main body.

5. The lancet device as set forth in claim 4, wherein the grip part is provided with a plurality of flat surface part, which is protruded along the outer circumferential surface of the grip part.
